(19) United States
(12) Patent Application Publication
(10) Pub. No.: US 2007/0060864 A1
(43) Pub. Date: Mar. 15, 2007

(54) Skin treatment method and system

(76) Inventor: Bruce K. Redding, Broomall, PA (US)

Correspondence Address: PLEVY & HOWARD & DARCY P.C., P.O. BOX 226, Fort Washington, PA, 19034, US

(21) Appl. No.: 11/435,273

(22) Filed: May 16, 2006

Related U.S. Application Data
(63) Continuation-in-part of application No. 11/130,817, filed on May 17, 2005.
(63) Continuation-in-part of application No. 10/949,044, filed on Sep. 24, 2004.
(63) Continuation-in-part of application No. 10/946,863, filed on Sep. 22, 2004.
(63) Continuation-in-part of application No. 10/848,519, filed on May 18, 2004.
(63) Continuation-in-part of application No. 10/782,378, filed on Feb. 18, 2004.
(63) Continuation-in-part of application No. 10/782,398, filed on Feb. 18, 2004.
(63) Continuation-in-part of application No. 10/345,825, filed on Jan. 16, 2003, now Pat. No. 6,908,448.
(63) Continuation-in-part of application No. 10/314,378, filed on Dec. 9, 2002.
(63) Continuation-in-part of application No. 09/939,507, filed on Aug. 24, 2001, now abandoned.
(63) Continuation-in-part of application No. 09/939,435, filed on Aug. 24, 2001, now abandoned.
(63) Continuation-in-part of application No. 09/939,506, filed on Aug. 24, 2001, now abandoned.
(60) Provisional application No. 60/681,572, filed on May 16, 2005.
(60) Provisional application No. 60/713,479, filed on Sep. 2, 2005.

(51) Int. Cl. A61B 17/20

(52) U.S. Cl. 604/22; 601/2

(57) ABSTRACT

A skin treatment device including: a housing; at least one ultrasonic transducer contained in the housing; a driver contained in the housing and electrically coupled to the at least one ultrasonic transducer and providing an excitation signal having alternating first and second waveform portions, wherein the first portion has an average imparted power greater than that of the second waveform portion; and, a cap detachably coupled to the housing and containing at least one substance to be delivered to the skin, the at least one substance being within a functional proximity to the transducers when the cap is attached to the housing; wherein, when the cap is attached to the housing and positioned adjacent to the skin, the at least one transducer emits ultrasound responsively to the excitation signal that impinges the substance and skin.

### RELATED APPLICATIONS

This application claims priority of: U.S. Patent Application Ser. No. 60/681,572, entitled SUBSTANCE DELIVERY DEVICE, filed May 16, 2005; U.S. patent application Ser. No. 60/713,479, entitled ULTRASONIC SUBSTANCE DELIVERY DEVICE FOR THE ADMINISTRATION OF COSMETIC, SKIN CARE AND PERSONAL CARE COMPOUNDS TO THE SKIN, filed Sep. 2, 2005; and is a continuation-in-part application of: U.S. patent application Ser. No. 11/130,817, entitled SUBSTANCE DELIVERY DEVICE, filed May 17, 2005; U.S. patent application Ser. No. 10/949,044, entitled ULTRASONICALLY ENHANCED SUBSTANCE DELIVERY SYSTEM AND DEVICE, filed Sep. 24, 2004; U.S. patent application Ser. No. 10/946,863, entitled ULTRASONICALLY ENHANCED SUBSTANCE DELIVERY SYSTEM, filed Sep. 22, 2004; U.S. patent application Ser. No. 10/848,519, entitled SYBSTANCE DELIVERY SYSTEM, filed May 18, 2004; U.S. patent application Ser. No. 10/782,378, entitled METHOD AND APPARATUS FOR THE MEASUREMENT OF REAL TIME DRUG DELIVERY THROUGH THE USE OF A WEARABLE MONITOR AND SENSOR ATTACHED TO A TRANSDERMAL DRUG DELIVERY DEVICE, filed Feb. 18, 2004; U.S. patent application Ser. No. 10/782,398, entitled ULTRASONICALLY ENHANCED SALINE TREATMENT FOR BURN DAMAGED SKIN, filed Feb. 18, 2004; U.S. patent application Ser. No. 10/345,825, entitled SUBSTANCE DELIVERY DEVICE, filed Jan. 16, 2003; U.S. patent application Ser. No. 10/314,378, entitled WEARABLE, PORTABLE SONIC APPLICATOR FOR INDUCING THE RELEASE OF BIOACTIVE COMPOUNDS FROM INTERNAL ORGANS, filed Dec. 9, 2002; U.S. patent application Ser. No. 09/939,507, entitled ULTRASONICALLY ENHANCED SUBSTANCE DELIVERY SYSTEM AND DEVICE, filed Aug. 24, 2001; U.S. patent application Ser. No. 09/939,435, entitled ULTRASONICALLY ENHANCED SUBSTANCE DELIVERY METHOD, filed Aug. 24, 2001; and U.S. patent application Ser. No. 09/939,506, entitled SUBSTANCE DELIVERY SYSTEM, filed Aug. 24, 2001; the entire disclosures of each of which are hereby incorporated by reference as if being set forth in their respective entireties herein.

### FIELD OF THE INVENTION

The present invention relates generally to skin care, and more particularly to skin treatment devices and methods.

### BACKGROUND OF THE INVENTION

Skin care and treatment substances are conventionally topically applied by rubbing them onto a user's skin. A simplified presentation of the structure of human skin is presented for non-limiting purposes of explanation in FIG. 1. However, those possessing an ordinary skill in the pertinent arts will recognize that skin, being the largest organ of the human body, possesses a unique and complicated structure. Referring to FIG. 1, skin is an organ of the integumentary system made up of layers of tissues. Skin is composed of the epidermis 20 and the dermis 30. The dermis 30 lies below the epidermis 20, and contains a number of structures, including blood vessels, nerves, smooth muscle, glands and lymphatic tissue. Below the dermis 20 lies the hypodermis. Skin structure 10 also includes hair follicles 40, that each extend from a root 50 through the stratum corneum 60, or outermost layer of skin structure 10. Sweat glands 60 open up via ducts onto the skin surface as a pore 70, i.e., a pore is an opening into a sebaceous gland that secretes oil to lubricate and protect the surface of the skin.

It has become commonplace to apply substances to the skin. One type of substance often applied to skin is a cosmetic. “Cosmetic”, as used herein, generally refers to a substance intended to enhance the beauty of the skin or human body. Non-limiting examples of cosmetics include cleansers and make-up. Non-limiting examples of make-up include, for example, foundation and powder, used to color the skin, and for lightening and concealing flaws, to produce an impression of health and youth and/or prevent skin from appearing too shiny. Another example of make-up is rouge, blush or blusher, used to color the cheeks and emphasize the cheekbones, for example. Other examples of cosmetics include, by way of non-limiting example only, skin moisturizers, skin creams, anti-wrinkle compositions, skin firming compositions, natural skin care compounds, toners, colorants, bath care compositions, anti-aging compounds, treatments for dry skin and treatments for skin spotting or discoloration.

Other substances often applied to the skin are medicaments. “Medicaments”, like cosmetics, may sometimes enhance the appearance of the skin. “Medicaments” may, alternatively or in addition thereto, be used to treat one or more skin conditions, such as to effect pore control and/or cleansing. One example of a skin treatment medicament is benzoyl peroxide. Another example of a medicament is sunscreen, sometimes referred to as sunblock, suncream or suntan lotion. A sunscreen is typically used to help protect the skin from the sun's ultraviolet radiation, and thus reduce sunburn and other skin damage. Other examples of medicaments include, by way of non-limiting example only, acne medication, treatments for skin abrasions, treatments for skin burns, lesions or abrasions, antiperspirants, dandruff treatments, treatments for other dermatological conditions, hair removal products and hair growth products. It should be understood that the present invention is directed to a broad number of substances suitable for delivery to the skin, including cosmetics and medicaments, and combinations thereof, for example.

While countless dollars and hours are spent developing, making, purchasing and applying various skin treatment substances, it is believed their efficacy is significantly limited due to conventional topical application techniques. Referring now also to FIG. 2, there is shown a cross-section ultrasonic view of skin structure 10. FIG. 2 is indicative of a control skin sample. The stratum corneum 60 and tissue structure of the dermis 30 may be seen therein. The internal structures of the skin reflect the imaging ultrasound signal and show up clearly at a depth of 3 mm.

Referring now also to FIG. 3, there is shown a cross-section ultrasonic view of skin structure 10 after the topical application of a conventional moisturizer cream by rubbing. As can be seen therein, it appears that the cream was not effectively delivered throughout the skin structure 10, instead residing mainly in the upper-most epidermis. That is, the stratum corneum 60 outer barrier of the skin is evident, and no penetration of the cream is observed below the stratum corneum 60 layer of the skin. The internal structures are reflecting the imaging ultrasound signal and still show up clearly at a depth of 3 mm. Thus, it is believed to be desirable to provide a method and system that effectively delivers substances into the skin, e.g., facilitates a substance penetrating into the skin—as is shown by way of non-limiting example in FIG. 4.

### SUMMARY OF THE INVENTION

A skin treatment device including: a housing; at least one ultrasonic transducer contained in the housing; a driver contained in the housing and electrically coupled to the at least one ultrasonic transducer and providing an excitation signal having alternating first and second waveform portions, wherein the first portion has an average imparted power greater than that of the second waveform portion; and, a cap detachably coupled to the housing and containing at least one substance to be delivered to the skin, the at least one substance being within a functional proximity to the transducers when the cap is attached to the housing; wherein, when the cap is attached to the housing and positioned adjacent to the skin, the at least one transducer emits ultrasound responsively to the excitation signal that impinges the substance and skin.

### BRIEF DESCRIPTION OF THE FIGURES

Understanding of the present invention will be facilitated by consideration of the following detailed description of the preferred embodiments of the present invention taken in conjunction with the accompanying drawings, in which like numerals refer to like parts and in which:

FIG. 1 illustrates a simplified view of the structure of human skin;

FIGS. 2-4 illustrate ultrasonic views of human skin under varying conditions;

FIGS. 5-9 illustrate various device configurations according to different embodiments of the present invention;

FIG. 10 illustrates a block diagrammatic view of the components of a device according to an embodiment of the present invention;

FIGS. 11A and 11B illustrate circuit diagrams according to embodiments of the present invention;

FIG. 12 illustrates a transducer excitation waveform according to an embodiment of the present invention;

FIG. 13 illustrates data indicative of human skin pore size under various conditions;

FIGS. 14 and 15 illustrate schematic representations of transducers according to embodiments of the present invention;

FIG. 16 illustrates a portion of a body of a device according to an embodiment of the present invention;

FIGS. 17 and 18 illustrate various views of a head of a device according to an embodiment of the present invention

FIGS. 19 and 20 illustrates various components of the head of FIGS. 17 and 18 according to an embodiment of the present invention; and,

FIG. 21 illustrates a head according to an embodiment of the present invention.

### DETAILED DESCRIPTION OF THE INVENTION

It is to be understood that the figures and descriptions of the present invention have been simplified to illustrate elements that are relevant for a clear understanding of the present invention, while eliminating, for purposes of clarity, many other elements found in typical skin treatment, and insonifying methods and systems. However, because such elements are well known in the art, and because they do not facilitate a better understanding of the present invention, a discussion of such elements is not provided herein. The disclosure herein is directed to all such variations and modifications known to those skilled in the art.

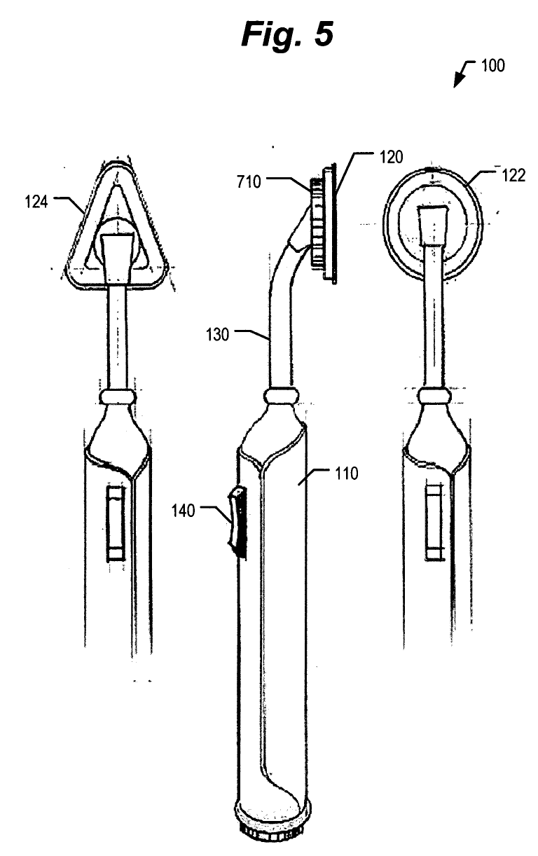

Referring now to FIG. 5, there are shown plan-views of a skin insonifying device 100 according to an embodiment of the present invention. Device 100 generally includes a body 110 and a head or cap 120. In the illustrated embodiment, body 110 is substantially cylindrical and has an elongated neck 130. However, it should be understood that body 110 may be configured in a wide variety of shapes and sizes. Likewise, two head 120 shapes are illustrated—a generally disc shaped head 122 and a substantially triangular shaped head 124. Again, however, it should be understood that head 120 may be configured in a wide variety of shapes and sizes.

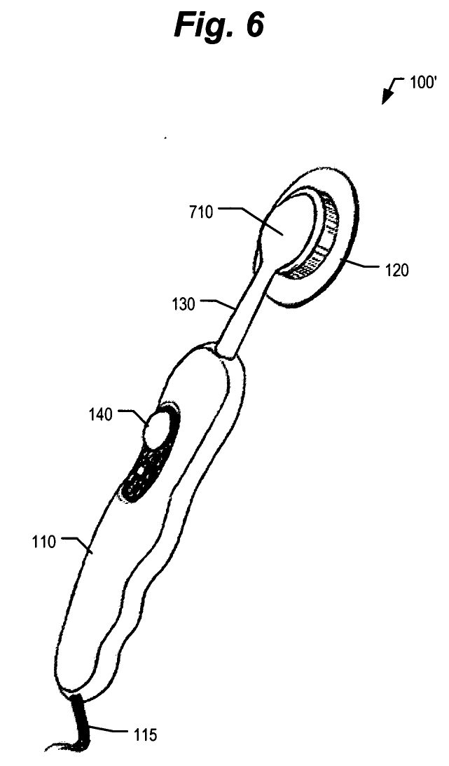

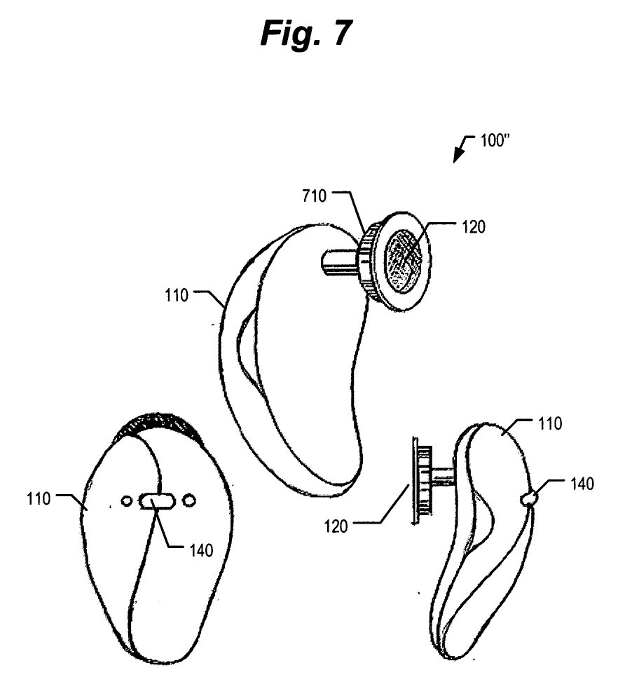

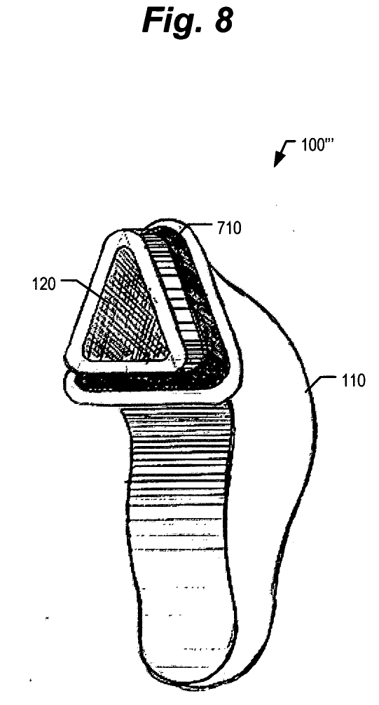

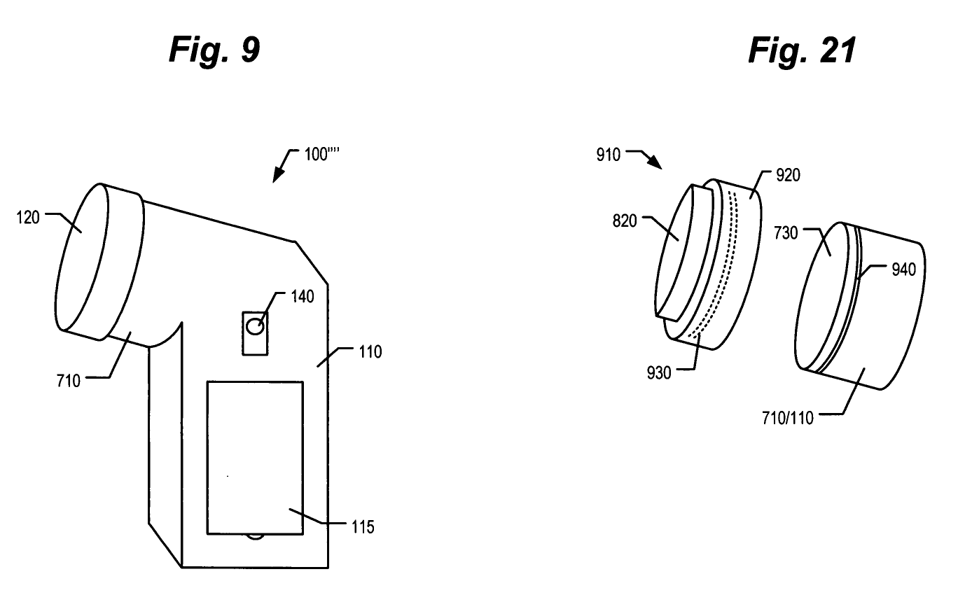

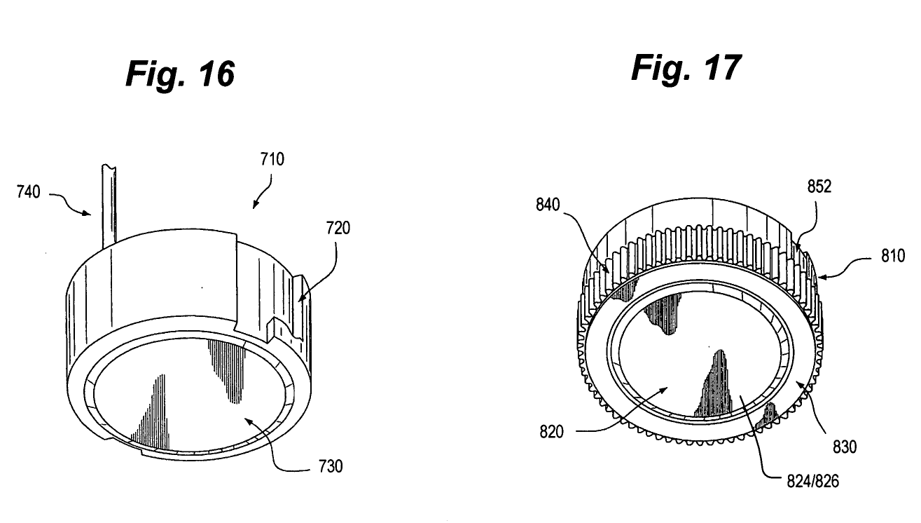

For example, FIG. 6 illustrates an alternative embodiment of the device, designated 100′. By way of further example, FIG. 7 illustrates an alternative embodiment of the device, designated 100″. By way of further example, FIG. 8 illustrates an alternative embodiment of the device, designated 100′″. And, by way of further example, FIG. 9 illustrates an alternative embodiment of the device, designated 100″″. The embodiment of FIG. 9 additionally includes a battery compartment cover 115. As is evident from FIGS. 7-9, devices 100″, 100′″, 100″″ do not include elongated necks. Portion 710 of the embodiments of FIGS. 7-9 are discussed with regard to FIGS. 16-20.

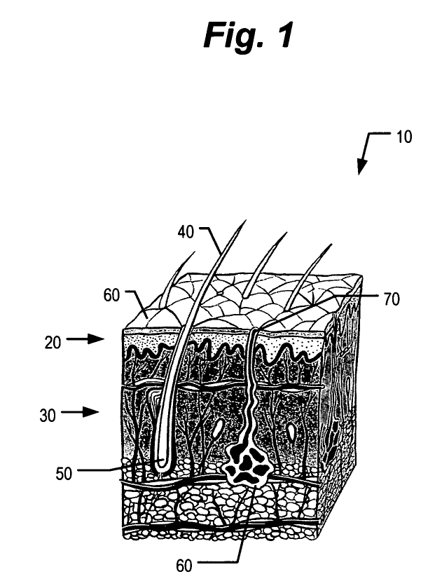

Referring still to FIGS. 5-9, head 120 is detachably coupled to body 110. Head 120 is adapted to be placed into contact with human skin, e.g., the stratum corneum 60 of skin structure 10 (FIG. 1). In one embodiment of the present invention, head 120 contains one or more substances to be delivered, e.g., one or more cosmetics and/or medicaments. Head 120 is adapted to deliver the one or more substances into the contacted skin structure responsively to activation of body 110. Body 110 is adapted to be activated by user interaction with activation device 140. In the illustrated embodiment, device 140 takes the form of a user selectable switch. Thereafter, head 120 is adapted to be removed from body 110, such that another head 120 containing additional substance(s) to be delivered may be connected to body 110.

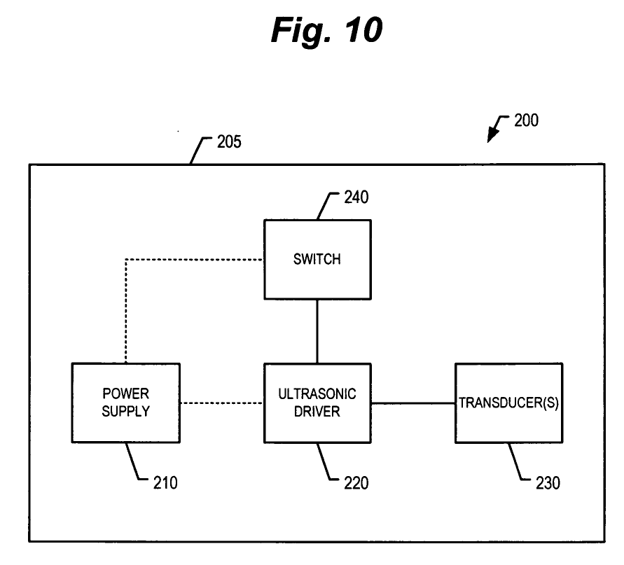

Referring now to FIG. 10, there is shown a block diagram of the components of a body 200 suitable for use as body 110 (FIGS. 5-9), according to an embodiment of the present invention. Generally, body 200 includes a housing 205 accommodating a power supply 210, ultrasonic transducer driver 220, one or more ultrasonic transducers 230 and one or more switches 240. Activation of switch 240, which may serve as activation device 140 of body 110 (FIGS. 5-9), activates driver 220 using power supply 210 to cause transducer(s) 230 to emit ultrasound—thereby insonifying head 120 contained substance(s) and an area of skin against which head 120 is placed.

Housing 205 may take any suitable size or shape. For example, in accordance with the embodiment of FIG. 9, housing 205 may be about 4 inches long, by about 1.5 inches wide and about 1 inch thick. Housing 205 may be formed of any suitable material, such as plastic and/or metal. For example, housing 205 may have a largely plastic body, with a metal diaphragm forming a sonic interface, or faceplate, between housing 205 and head 120.

Power supply 210 may take the form of a battery and/or voltage regulator, for example. Where a battery is used, a commercially available 9-volt battery may be particularly well suited for use. Alternatively, a conventional rechargeable battery configuration may be used. In lieu of, or in addition to a battery, a conventional voltage regulator may be used. Further, a conventional power cord (as is designated 115 in FIG. 6, for example) may extend from housing 205. Optionally, one or more voltage regulator components may be provided external to the housing 205, electrically in-line with the power cord, such as a conventional AC/DC converter power pack.

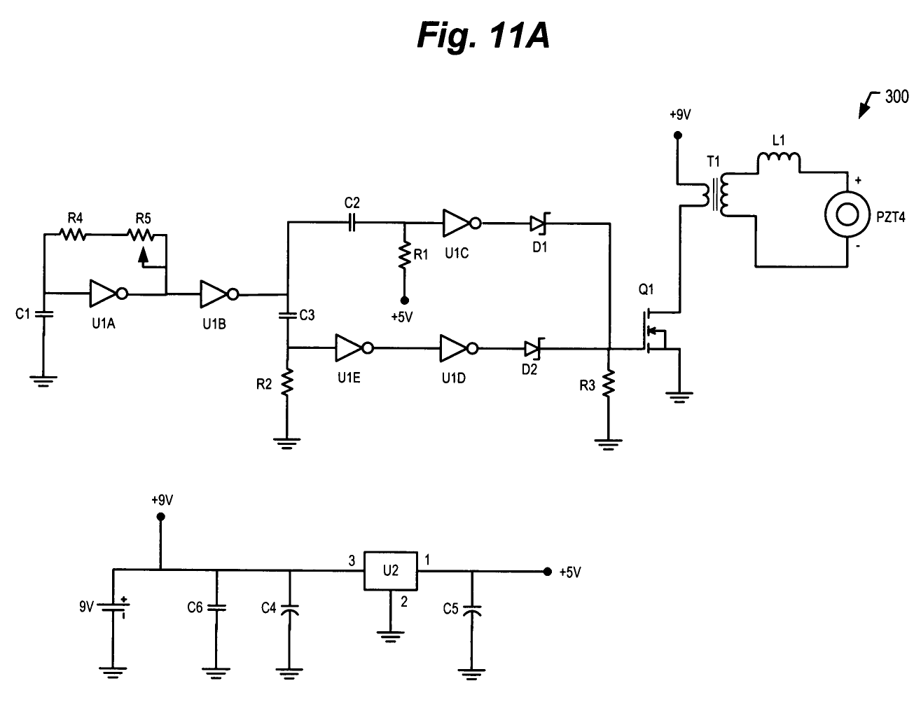

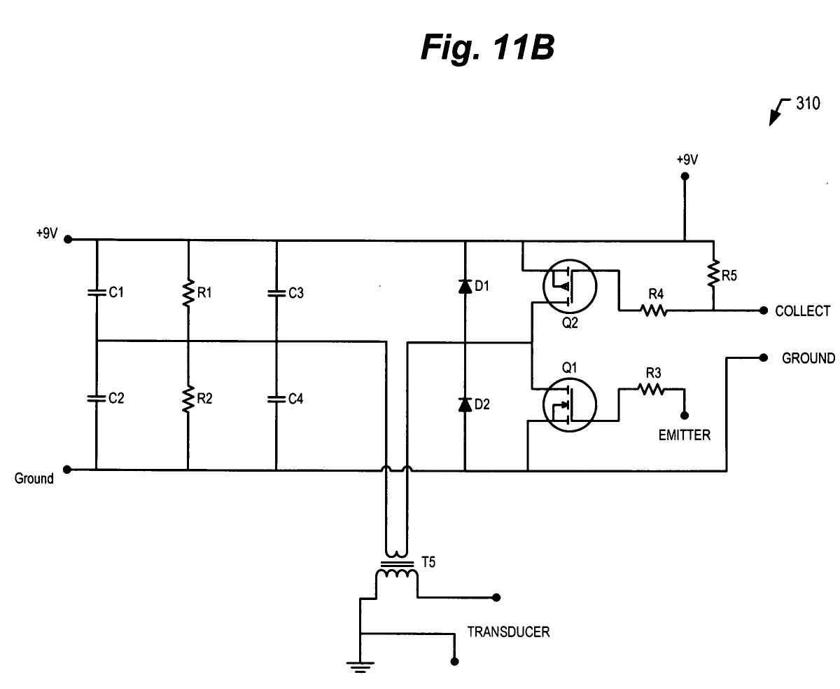

Referring still to FIG. 10, ultrasonic driver 220 may take the form of circuitry being suitable for driving, e.g., activating, transducer(s) 230 responsively to switch 240. Driver 220 and power supply 210 may take the form of the circuitry illustrated in FIG. 11A, where a single transducer is used, for example. For non-limiting purposes of explanation only, all referenced circuit parts are available through Digi-Key Corporation. FIG. 11A shows a circuit 300. Circuit 300 includes a conventional 9-volt battery, which provides a +9V supply voltage and is coupled between ground and a first terminal (designated 3) of a quasi LDO voltage regulator U2, such as part no. LM34801M3-5.0CT-ND. The first terminal of the voltage regulator is also coupled to ground through a 0.1 μF capacitor C6 and a 33 μF capacitor C4, in parallel. A second terminal of the regulator (designated 2) is coupled to ground. A third terminal (designated 1) of regulator U2 provides a +5V supply voltage and is coupled to ground through a 33 μF capacitor C5.

The +9V supply is provided to a first terminal of a first winding of a step-up transformer T1, that may take the form of a part no. 179-2026ND, for example. The second winding of transformer T1 has a first terminal inductively coupled to the transducer (e.g., 230, FIG. 10) serially through a 6800 μH inductor L1. A second terminal of the second winding is directly coupled to the transducer. A second terminal of the first winding of transformer T1 is coupled to a first terminal of a PNP transistor Q1, that may take the form of part no. MMBT3906LT10SCT-ND. A second terminal of transistor Q1 is coupled to ground. A third terminal of transistor Q1 is resistively coupled to ground through a 2 kΩ resistor R3. The third terminal of transistor Q1 is also coupled to 5.6 volt Zener diodes D1, D2, that may each take the form of part no. DDZX5V6BDICT-ND. Diodes D1, D2 are, in turn, respectively coupled to the outputs of Hex CMOS Schmidt Triggers U1C, U1D, that may each take the form of part no. CD74HC14M.

A second terminal of trigger U1C is coupled: to the +5V supply through an 18 KΩ resistor R1; and to ground serially through a 270 pF capacitor C2, a 27 pF capacitor C3 and an 18 kΩ resistor R2. Another Hex CMOS Schmidt Trigger U1E is coupled between a common node between capacitor C3 and resistor R2, and a second terminal of trigger U1D. Another Hex CMOS Schmidt Trigger U1B is coupled between a common node between capacitors C2, C3 and another Hex CMOS Schmidt Trigger U1A. Trigger U1B is also coupled to another terminal of trigger U1A serially through a potentiometer R5 and 51 kΩ resistor R4, which is in turn also coupled to ground through a 0.001 μF capacitor C1. Additionally, a conventional timer may be included to limit circuit operation to a given temporal period per user activation, for example to a given number of seconds, after which the excitation of the transducer(s) 230 is automatically ended. Further, a user feedback device, such as a visible or audible indicator, that indicates that the driver is exciting the transducer(s) may also be provided.

Referring now also to FIG. 11B, where two transducers are to be used, circuit 310 may also be used according to an embodiment of the present invention. Circuit 310 also is fed by the +9V supply, which is, in parallel: capacitively coupled to ground serially through 1 μF capacitors C1, C2; resistively coupled to ground serially though 100 kΩ resistors R1, R2; capacitively coupled to ground serially through 1 μF capacitors C3, C4; to ground serially through diodes D1, D2, that may each take the form of part no. 2N4936; and to ground through transistors Q1, Q2. Transistor Q1 may take the form of a part no. IRFZ34, where Q2 takes the form of a part no. IRF5305.

A common node between capacitors C1, C2, resistors R1, R2 and capacitors C3, C4 ,is coupled to a first terminal of a first winding of a step-up transformer T5, that may take the form of a part no. 179-2026 ND, for example. The second winding of transformer T5 is coupled across up to four (4) transducers in parallel, with one of the terminals thereof being grounded.

The second terminal of the first winding of transformer T5 is coupled to a common node between diodes D1, D2 and transistors Q2, Q1. A third terminal of transistor Q1 is coupled to a 20 Ω resistor R3. A third terminal of transistor Q2 is coupled to a 20 Ω resistor R4. A collector terminal is also coupled to resistor R4, and to the +9V supply though a 1 kΩ resistor R5. An emitter terminal is coupled to resistor R3. The collector and ground terminals may be coupled to the collector and emitter terminals of a NPN transistor (not shown), that may take the form of part no. LM3524DN. The +9V and emitter terminals may be coupled to the collector and emitter terminals of another NPN transistor (not shown), that may take the form of part no. LM3524DN. These NPN transistors may be used for system analysis, with an oscilloscope, for example.

Referring again to FIGS. 5-10, where a head 120 is attached to a body 110, transducer(s) 230 are positioned substantially adjacent to head 120, such that when head 120 is placed against a skin structure 10 (FIG. 1), and the transducer(s) 230 are activated, one or more substances contained within head 120 are delivered into the skin. That is, ultrasonic transmissions from transducer(s) 230 induce and/or aid the delivery of the one or more substances within head 120 to skin structure 10.

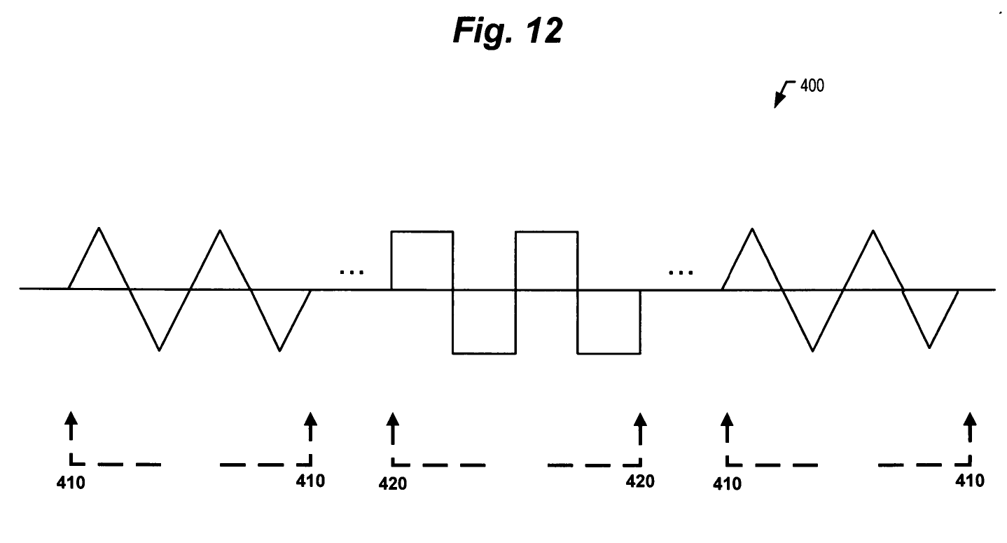

According to an embodiment of the present invention, driver 220 may excite transducer(s) 230 using an alternating waveform signal—thus activating and causing the transducer(s) to emit ultrasound. According to an embodiment of the present invention, analogous ultrasonic signaling, and signal shaping, may result. Using such an alternating waveform approach allows the amount of energy transmitted to the surface of the skin to be reduced while still providing a pressure wave effect upon the skin, by enhancing substance delivery through the hair follicle and pore system (see, e.g., 40/70, FIG. 1). Referring now also to FIG. 12, according to an aspect of the invention, driver 220 may excite transducer(s) 230 using a signal 400 that includes alternating at least first and second portions 410, 420, wherein the waveform envelope of the first portion 410 is distinct from the waveform envelope of the second portion 420. In the non-limiting illustrated case of FIG. 12, the first portion 410 takes the form of a substantially sawtooth waveform and the second portion 420 takes the form of a substantially square waveform. It should be understood, however, that other varying waveforms having alternating average imparted powers, and/or power profiles for example, can be utilized.

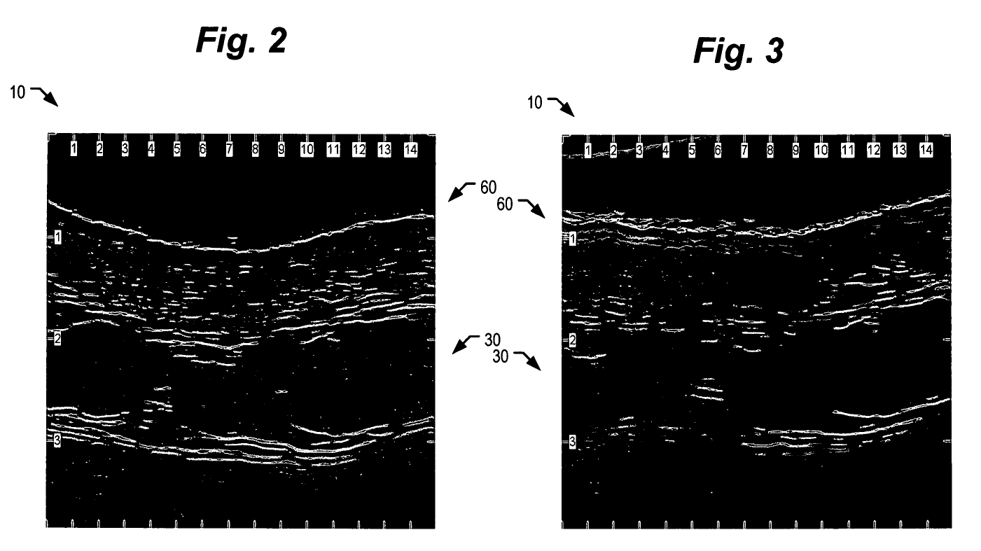

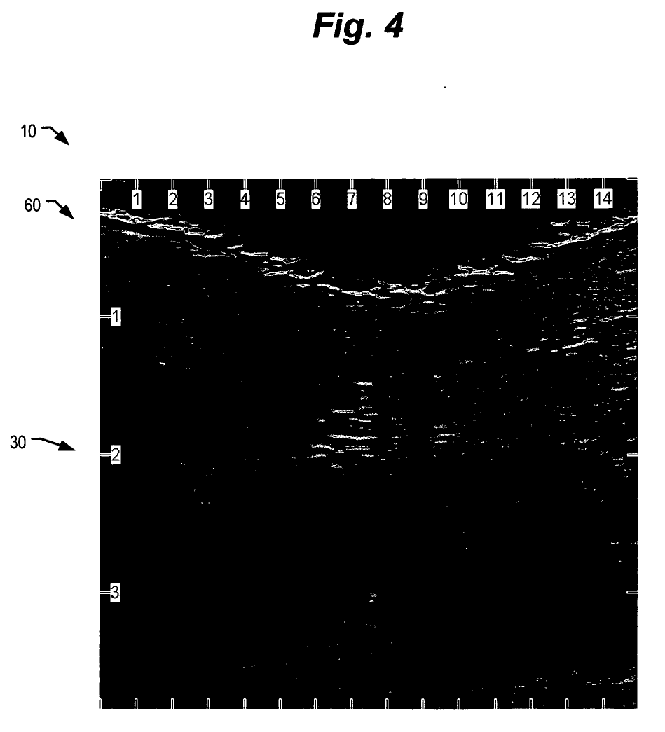

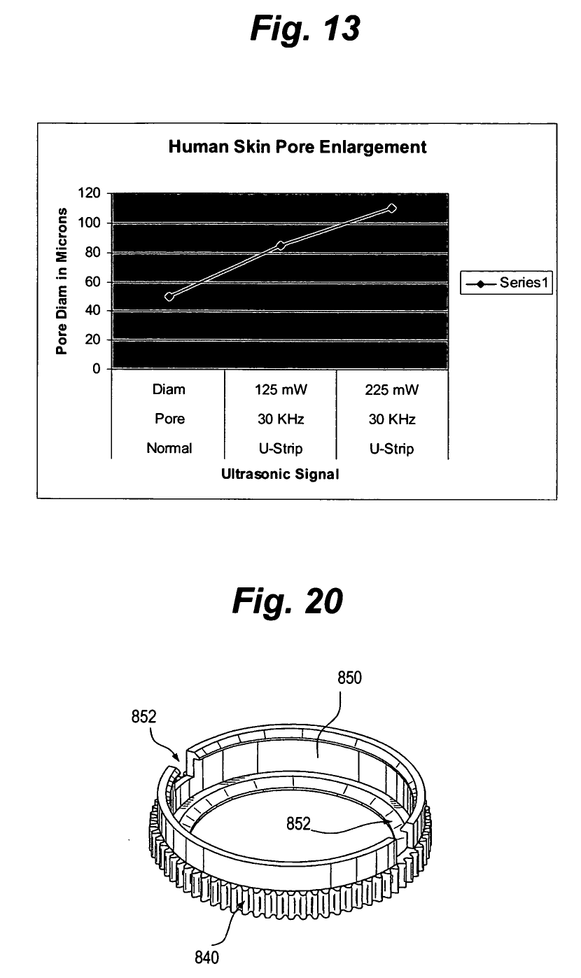

While not limiting the present invention, it is believed that the short, sawtooth peaks induced by the sawtooth shaped input portion 410 impart little heat to the substance(s) to be delivered, but aid their mixing and homogenization. These short peaks of the ultrasonic pressure waves also are believed to help with skin permeability. That is, the sawtooth excitation resulting ultrasound is believed to massage and open the fatty tissue surrounding hair follicles and pores. For example, it is believed that hair follicle openings that are normally around 50 μm in diameter may relax to around 110 μm in diameter. It is believed this effect can be seen in FIG. 4, as the vertical darker lines, as compared to FIGS. 2 and 3 where such an effect cannot be seen. This effect is further illustrated in FIG. 13, where it may be seen therein that the pores of skin structure 10 (FIG. 1) enlarge with insonification. At rest, the normal pore diameter is estimated at about 50 μm. Under insonification of the skin responsively to a sawtooth excitation at 30 KHz, and 125 mW/cm², the pore diameter is expected to enlarge to about 85 μm. Consistently, under insonification at 30 KHz, and 225 mW/cm², the pore diameter is expected to enlarge to about 110 μm. Accordingly, ultrasound emitted responsively to the sawtooth portion may be seen to enhance substance delivery through the hair follicle and pore system. In turn, the square waveform portion 420 (FIG. 12) may help to “push” the one or more substances through the pores and alongside the hair follicles, such that they permeate into skin structure 10 (FIG. 1).

Various ultrasound frequencies, intensities, amplitudes and/or phase modulations may be used to control the magnitude of the substance delivery into the skin. By way of non-limiting example only, a suitable excitation may be used to generate ultrasonic transmissions having a frequency on the order of 16-20 kHz, and an intensity around 125 mW/cm² to around 225 mW/cm². Alternatively, higher operating frequencies, such as greater than 20 kHz, or between 20 kHz and 175 Khz, or even up to 1 MHz may be used. Further, by varying the duty cycle of the first and second excitation portions (e.g., the percent of the time the first waveform portion excitation is present as compared to the percent of the time the second waveform portion is present), the relative depth of resulting substance permeation into the skin may be controlled. For example, it is believed that a duty cycle reflective of about 80% of the excitation time being of a sawtooth portion and about 20% of the excitation time being of a square waveform portion will result in about 90% of substance delivery into the outermost two or three millimeters of the skin structure when an around 18-19 KHz excitation signal having an intensity of about 125 mW/cm² is applied. In such a case, the sawtooth portion 410 may be used to excite the transducer(s) 230 for around 80 msec, followed by the square waveform portion 420 for around 20 msec, when the sawtooth waveform is again applied, and so on. It is also believed that increasing the duty cycle of the square waveform portion will increase the depth of substance penetration, and decreasing the square waveform portion will likewise decrease substance penetration.

Referring again to FIG. 10, transducer(s) 230 may take the form of one or more ultrasonic transducers. Transducer(s) 230 may take the form of a cymbal transducer, or array thereof. A stacked array of such transducers may be used to deliver low frequency ultrasound for dermal substance delivery and therapeutic applications responsively to driver 220. U.S. Pat. No. 5,729,077, issued Mar. 17, 1998, entitled “METAL-ELECTROACTIVE CERAMIC COMPOSITE TRANSDUCER” (Newnham et al.), the entire disclosure of which is incorporated by reference herein, discloses the use of stacked transducers, essentially transducers fitted atop each other, to increase ultrasonic intensities while maintaining a given frequency level. A stacked transducer construction may be used to increase intensity while improving the power utilization of the transducer system.

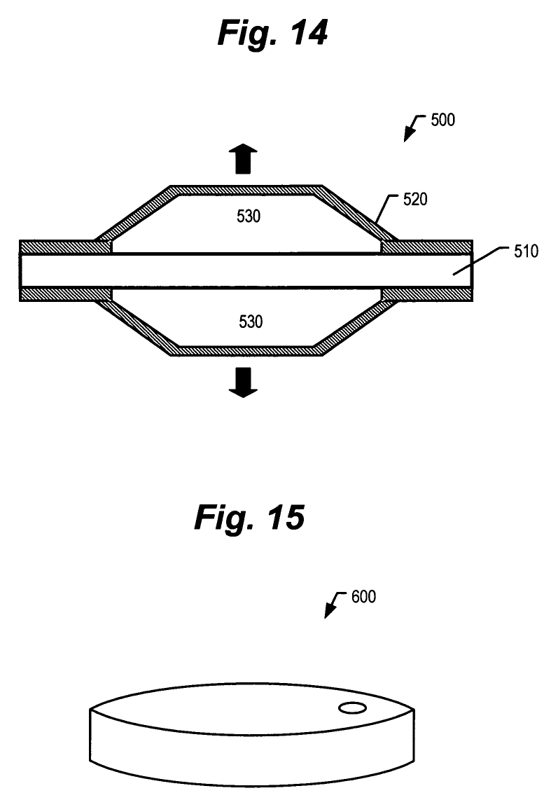

Referring now also to FIG. 14, transducer(s) 230 may take the form of one or more Class V flextensional cymbal type ultrasonic transducers 500. Such a cymbal transducer may include a piezoelectric disc 510, such as a PZT-4 disc connected between two metal caps 520, which may be composed of titanium foil for example. FIG. 14 illustrates hollow air spaces 530 between the piezoelectric disc 510 and the end caps 520. The end caps 520 are connected to the piezoelectric disc 410 by a non-electrically conductive adhesive 540, to form the bonded layered construction of transducer 500.

More particularly, two disks of about 0.25 mm thick, metal basis 5%, titanium foil from Alfa Aesar, A Johnson Matthey Company, of Ward Hill, Mass. may be cut using a circular saw having an about 10.7 mm diameter. Any resulting rough edges may be removed, by sanding for example. The foil disks may then be cleaned using alcohol. Each disk may then be pressed into the desired shape (shown in FIG. 14). Rough edges may again be removed, such as by sanding. An alcohol cleaning and subsequent drying may again be carried out. Pressed foils having nearly matching thicknesses may then be paired up—as caps having sufficiently different thicknesses may result in spurious resonances within the finished transducer during operation. The disks may again be cleaned. Thereafter, a ring of bonding epoxy may be screen printed onto each pair of matching disks. The epoxy printed titanium caps may then be pressed against the two surfaces of a 0.5-inch diameter, 1-mm thickness PZT-4 disk, which is commercially available from Piezo Kinetics Inc., of Bellefontte Pa. The combination may be held in place, such as by clamping, and then heated to around 70 degrees Celsius for around 4 hours or longer. Thereafter, the edges of the titanium caps may be soldered, at around 270 degrees Celsius or less, to the ceramic disk at four points, for example. Multiple such transducers can be stacked and affixed to one another to form a stacked array.

Alternatively, transducer(s) 230 may take the form of 2 or more transducers of the type shown in FIG. 15. FIG. 15 illustrates the structure of a transducer 600. Transducer 600 takes the form of an about 0.25 inches diameter by 1 mm thick lead zirconate titanate (PZT-4) disc, and may have a resonant frequency less than 20 kHz, such as around 18.75 kHz. An operating wattage may be on the order of 17-20 W, and a corresponding output intensity may be on the order of 125 mW. The electrical capacitance of such a device may be on the order of about 1.2 nF.

Two such transducer(s) 600 may be adhesively affixed to a metal diaphragm, i.e., sonic interface or faceplate, suitable for passing ultrasonic emissions therefrom into head 120 (FIGS. 5-9). Such a diaphragm may be formed of 316 stainless steel having a thickness around 0.1 mm, for example. One example of suitable adhesive is a 45LV black epoxy resin, which is commercially available from Emerson & Cuming, a National Starch & Chemical Company, cross-linked with a catalytic hardener, such as EC600505, which is also available from Emerson & Cuming. Any suitable adhesive that does not unduly reflect the ultrasonic transmissions may be used though.

Referring again to FIG. 10, switch 240 may take the form of any user-selectable switching device. For example, switch 240 may take the form of a simple on/off switch. In one embodiment, switch 240 is electrically coupled between power supply 210 and driver 220. In such a configuration, switch 240 may simply operate to selectively connect driver 220 to power supply 210 when user activated. In another embodiment, switch 240 may be connected to driver 220, such that driver 220 selectively excites transducer(s) 230 responsively to user activation of switch 240.

Referring again to FIGS. 5-9, head 120 may be pre-loaded with one or more substances to be delivered. The substance(s) may be applied to a fabric or absorbent pad that forms at least part of head 120. Alternatively, or in addition thereto, head 120 may be adapted to have one or more substances applied by a user to such a fabric or absorbent prior to use thereof.

Referring now to FIG. 16, there is shown a portion 710 of housing 110 (see e.g., FIG. 9) adapted to receive head 120 according to an embodiment of the present invention. Portion 710 includes at least one connector groove 720. Portion 710 also includes a metal face plate 730, to an interior surface of which transducer(s) 230 (FIG. 10) are affixed, that forms a sonic interface with head 120 (FIGS. 5-9). An electrical lead or cable 740 may be used to connect the transducer(s) 230 with the power source 210 and/or a driver 220.

Referring now to FIG. 17, there is shown a head 810 suitable for use with portion 710 of FIG. 16 as head 120 (FIGS. 5-9). Head 810 may be constructed of plastic or any sonically compatible material. Head 810 may include an absorbent fiber or pad 820, which contains the at least one substance to be delivered. According to an aspect of the invention, the absorbent 820 may be constructed of a cellulose pad, including wood pulp with ethylene vinyl acetate-based synthetic latex, such as Vicell #6009, available from Buckeye Absorbent Products, Memphis, Tenn. Many other materials are suitable for use as well though.

For example, the fabric or absorbent pad may include one or more of the following: cellulose fiber, cotton, natural sponge, woven cloth fabrics, polyurethane foams, polyisocynurate foams, non-woven cloths, fumed silica, starch, corn meal, wood pulp fibers, collagen pads, poly methyl methacrylate, polyvinyl alcohol, poly vinyl pyrrolidine, poly acrylic acid, poly(2)-hydroxy ethyl methacrylate, polyacrylamide, poly ethylene glycol, polylactides(PLA), polyglycolides(PGA), poly(lactide-Co-glycolides), polycarbonate, chitosan, poly (N-isopropylacrylamide), co-Polymer formulations of poly methacrylic acid and poly ethylene glycol, co-polymer formulations of poly acrylic acid and poly (N-isopropylacrylamide), hyrdogels, e.g. polyacrylamide, poly(propylene oxide), pluronic polyols family of gel materials, e.g. pluronic-chitosan hydrogels, silica gels, and other natural or synthetic material, that act to absorb the one or more substances and thereafter release them upon ultrasonic excitation.

Alternatively, no pad need be used, where the at least one substance to be delivered may be otherwise contained where pad 820 would otherwise be. In other words, head 810 may define an interior cavity that houses or contains the at least one substance to be delivered—optionally in an absorbent material or pad.

Referring again to FIG. 17, head 810 may optionally include a ring 830 suitable for contacting the skin of a subject and forming a seal substantially preventing the entry of air or contaminants under or into the device and the leakage or contamination of a substance deposited upon the skin of a subject by the delivery device. Optionally, ring 830 may be formed of a plastic or metal well suited for being glided over the surface of a user's skin.

Head 810 may also include a textured surface 840 on an outer surface to improve a user's ability to grip and turn head 810. According to an embodiment of the invention, head 810 may be disposable, such that after the substance is dispensed therefrom, head 810 may be discarded and replaced with a fresh head 810. Alternatively, pad 820 may be adapted to be replaced without requiring a new head 810. Either way, additional substance to be delivered may be provided without replacing the body 110 (FIGS. 5-9). Of course, the device may be designed such that all or part of body 110 (FIGS. 5-9) may be replaced periodically, or the device may be designed as a single-use device.

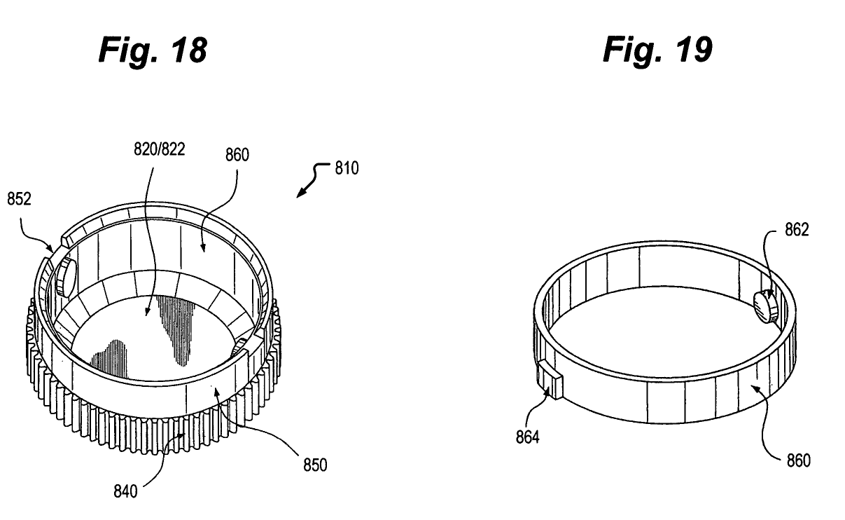

Referring now to FIG. 18, head 810 may be constructed of an outer snap ring 850 and an inner snap ring 860 nested inside of the outer snap ring 850. Rings 850, 860 may be formed of sonically compatible material such as plastic, for example. According to an embodiment of the invention, the inner snap ring 860 may substantially secure the absorbent pad 820 in place. Referring now also to FIGS. 19 and 20, the inner snap ring 860 may include at least one inner connector tab or bayonet 862 and at least one outer connector tab 864. The outer snap ring 850 includes at least one connector groove 852 into which the outer connector tab 864 of the inner snap ring 860 may be inserted so as to screw couple the inner snap ring 860 to the outer snap ring 850. The inner connector tab 862 may be fitted into the portion 710 connector groove 720 (FIG. 16) to allow a tight connection when the head 810 is screw coupled with the portion 710. According to an aspect of the invention, the connection between the head 810 and the portion 710 may form a sonic connection between the absorbent pad 820 and the transducers(s) 230 within portion 710. According to an aspect of the invention, portion 710 may include two transducers, side-by-side. In such a configuration, the absorbent pad 820 may be secured substantially adjacent to the portion 710, and/or faceplate 730.

A membrane 822 may be provided that covers the absorbent pad 820 on a side which is placed in contact with the metal face plate 730. According to an aspect of the invention, the membrane may be constructed of polyvinylidene chloride plastic film, such as, for example, the film sold under the trademark Saran and model number Dow BLF-2014, available from Dow Chemical company, Midland, Mich. According to an embodiment of the invention, the membrane may have a thickness of about 50 μm. Alternatively, membrane 822 may be constructed of polyester film, for example, a Mylar film, including, but not necessarily limited to, model number M34, available from DuPont Teijin Films Div., Wilmington, Del. According to an aspect of the invention, the polyester membrane may have an about 13 μm thickness.

A peel-away film 824 may be secured over head 810 such that prior to the removal thereof, it covers a side of the absorbent pad 820 that is to be placed against the skin of a subject. Upon removal of peel-away film 824, absorbent pad 820 containing the at least one substance may be exposed so as to be secured adjacent to the skin. Where no absorbent pad is utilized, the membrane 822 and peel-away film 824 may be used to contain the at least one substance to be delivered within head 810.

According to an embodiment of the invention, head 810 may optionally include a semi-permeable membrane 826 (FIG. 17) positioned between the absorbent pad 820 and on the underside of head 810—adapted to be positioned against a user's skin, such that the semi-permeable membrane comes into functional proximity with the surface of a user's skin. The semi-permeable membrane may be constructed of any sonically suitable material, including, but not necessarily limited to, ethylene-co-methacrylic acid copolymers (such as, for example, the film sold as Surlyn, which is available from DuPont, of Wilmington, Del.

Alternatively, and referring now also to FIG. 21, there is shown another head 910. Head 910 is illustrated in combination with the portion 710 of embodiment 100″″ of FIG. 9 for non-limiting purposes of explanation only. Head 910 generally includes two portions, portion 920 and absorbent pad 820, analogous to that described herein-above. Portion 920 is adapted to slide over at least part of portion 710 of body 110. One or more conventional mechanism for assisting the placement, and/or retaining of portion 920 over/on portion 710 of body 110 may be provided. In the illustrated case, portion 920 is provided with one or more internal ridges 930, while portion 710 is provided with one or more mating external ridges 940. In this manner, portion 920 may be force- or press-fit onto portion 710, such that face plate 730 is sonically coupled through region 920 to pad 820. Portion 920 may take the form of any suitable material, including by way of non-limiting example only, plastic, metal and/or acoustic transmitting foam. One example of a suitable foam is a polyurethane or polyisocynurate foam that allows sonic transmissions to traverse from portion 710 to pad 820. Optionally, such a material may provide a sponge-like function for a substance including cream, solution or gel. It should also be understood that while pad 820 is illustrated to have smaller lateral dimensions than, and longitudinally extend from, portion 920 by way of non-limiting example only. The device of FIGS. 5-9 may be constructed such that the head 120 and the body 110 may be press-fit, screwed or snapped together to form a dermal delivery device or assembly.

Referring finally now to FIG. 4, there is shown an ultrasonic image of skin structure 10 demonstrating improved moisturing cream permeation. These results were achieved by rubbing the moisturizing cream into the skin, and then applying ultrasound from four 125 mW/cm2 transducers at 23 kHz, with a 500 msec sawtooth followed by a 500 msec square wave signal envelope for 10 seconds. The image therein shows the internal structures as virtually non-reflective. This means the imaging signal transmitted easily through the skin internal structures, instead of echoing back to the imaging device. On the scan it shows up as if the internal skin structures have disappeared, but this is not the case. By way of non-limiting example only, it is surmised that the moisturizer cream has simply lubricated the internal skin tissue and thereby mitigates the echo pattern. The cream shows up as a purely black or void image in the scan.

It will be apparent to those skilled in the art that modifications and variations may be made in the apparatus and process of the present invention without departing from the spirit or scope of the invention. It is intended that the present invention cover the modification and variations of this invention provided they come within the scope of the appended claims and their equivalents.

What is claimed is:

1. A skin treatment device comprising:
a housing;
at least one ultrasonic transducer contained in said housing;
a driver contained in said housing and electrically coupled to said at least one ultrasonic transducer and providing an excitation signal having alternating first and second waveform portions, wherein the first portion has an average imparted power greater than that of the second waveform portion; and,
a cap detachably coupled to said housing and containing at least one substance to be delivered to the skin, said at least one substance being within a functional proximity to said transducers when said cap is attached to said housing;
wherein, when the cap is attached to the housing and positioned adjacent to the skin, said at least one transducer emits ultrasound responsively to said excitation signal that impinges the substance and skin.

2. The device of claim 1, wherein said housing is of a hand-held size.

3. The device of claim 1, wherein the at least one transducer operates at between about 20 kHz and 175 kHz.

4. The device of claim 1, wherein the at least one transducer operates at less than 20 kHz.

5. The device of claim 4, wherein the at least one transducer operates at between about 18 kHz and 19 kHz.

6. The device of claim 5, wherein the at least one transducer operates at about 18.75 kHz.

7. The device of claim 1, wherein the at least one transducer operates at between about 175 kHz and 1 MHz.

8. The device of claim 1, where each at least one transducer has an operating intensity between about 0.01 and 5.0 W/cm².

9. The device of claim 8, wherein each said at least one transducer has an operating intensity between about 100 and 250 mW/cm².

10. The device of claim 9, wherein each said at least one transducer has an operating intensity about 125 mW/cm² during the first waveform portion.

11. The device of claim 11, wherein each said at least one transducer has an operating intensity between about 225 mW/cm² during the second waveform portion.

12. The device of claim 1, further comprising a timer, wherein the driver automatically ceases exciting the at least one transducer responsively to the timer.

13. The device of claim 12, wherein said timer has a duration greater than 1 second.

14. The device of claim 1, further comprising a battery contained in the housing.

15. The device of claim 14, wherein the battery is rechargeable.

16. The device of claim 1, wherein the at least one transducer comprises at least one cymbal transducer.

17. The device of claim 1, wherein the at least one transducer comprises a PZT-4 disc.

18. The device of claim 1, wherein the at least one transducer comprises an array of transducers.

19. The device of claim 18, wherein the array is a 1×2 array.

20. The device of claim 18, wherein the array comprises at least two stacked transducers.

21. The device of claim 1, wherein the cap defines an interior cavity containing the at least one substance.

22. The device of claim 21, further comprising at least one detachable film closing at least a part of the cavity.

23. The device of claim 21, further comprising at least one film closing at least a part of the cavity.

24. The device of claim 1, further comprising at least one absorbent material containing the at least one substance.

25. The device of claim 1, wherein a duty cycle between the first and second waveform portions is greater than about 2:1.

26. The device of claim 1, wherein a duty cycle between the first and second waveform portions is about 4:1.

27. The device of claim 1, wherein insonifying the skin responsively to the first waveform portion effects a largening of pores.

28. The device of claim 1, wherein insonifying the at least one substance responsively to the second waveform portion effects a transport of at least a part of the at least one substance into the skin.

29. The device of claim 1, wherein the cap comprises a plastic housing containing an absorbent pad.

30. The device of claim 1, wherein the cap comprises a foam backing and an absorbent pad.

31. A method for treating an area of a subject's skin comprising:
positioning at least one substance at least substantially adjacent to the area of skin;
insonifying the at least one substance and area of the skin with a first ultrasonic signal having substantially sharp temporal peaks in power; and,
insonifying the at least one substance and area of the skin with a second ultrasonic signal having substantially broad peaks in power;
wherein, at least a portion of the at least one substance is delivered into the area of skin.

32. The method of claim 31, wherein the insonifying comprising gliding at least one transducer containing housing over the area of skin.

33. The method of claim 32, wherein the at least one transducer operates at between about 20 kHz and 175 kHz.

34. The method of claim 32, wherein the at least one transducer operates at less than 20 kHz.

35. The method of claim 34, wherein the at least one transducer operates at about 18.75 kHz.

36. The method of claim 32, wherein the at least one transducer operates at between about 175 kHz and 1 MHz.

37. The method of claim 32, where each at least one transducer has an operating intensity between about 0.01 and 5.0 W/cm².

38. The method of claim 37, wherein each said at least one transducer has an operating intensity between about 100 and 250 mW/cm².

39. The method of claim 38, wherein each said at least one transducer has an operating intensity about 125 mW/cm² during the first waveform portion.

40. The method of claim 39, wherein each said at least one transducer has an operating intensity between about 225 mW/cm² during the second waveform portion.

41. The method of claim 31, further comprising automatically ceases the insonifying after a predetermined time period.

42. The method of claim 41, wherein the time period is between around 10 and 25 seconds.
